(19) United States
(12) Patent Application Publication
(10) Pub. No.: US 2023/0338707 A1
(43) Pub. Date: Oct. 26, 2023

(54) Indicators for Instrument Advancement Device

(71) Applicant: Becton, Dickinson and Company, Franklin Lakes, NJ (US)

(72) Inventors: Curtis H. Blanchard, Herriman, UT (US); John M. Lackey, West Valley City, UT (US); Megan S. Scherich, Salt Lake City, UT (US); Benjamin P. Hopwood, West Valley City, UT (US); Yiping Ma, Layton, UT (US)

(21) Appl. No.: 18/135,808

(22) Filed: Apr. 18, 2023

Related U.S. Application Data
(60) Provisional application No. 63/333,660, filed on Apr. 22, 2022.

(51) Int. Cl. A61M 25/01

(52) U.S. Cl. CPC A61M 25/0113; A61M 2025/0008

(57) ABSTRACT

An instrument advancement device for advancing an instrument through a vascular access device includes a housing having a distal end and a proximal end, with the distal end configured to couple to a vascular access device, an instrument at least partially received within the housing, with the instrument having an advanced position where a distal end of the instrument extends beyond the distal end of the housing and a retracted position where the distal end of the instrument is received within the housing, an actuator configured to move the instrument between the advanced position and the retracted position, and an indicator configured to be visible only when the instrument is in the advanced position.

### CROSS-REFERENCE TO RELATED APPLICATION

The present application claims priority to U.S. Provisional Application Ser. No. 63/333,660, entitled “Indicators for Instrument Advancement Device”, filed Apr. 22, 2022, the entire disclosure of which is hereby incorporated by reference in its entirety.

### BACKGROUND OF THE INVENTION

### Field of the Invention

The present disclosure generally relates to indicator arrangements for an instrument advancement device.

### Description of Related Art

A catheter is commonly used to infuse fluids into the vasculature of a patient. For example, the catheter may be used for infusing normal saline solution, various medicaments, or total parenteral nutrition. Furthermore, the catheter may also be used for withdrawing blood from the patient.

The catheter may be an over-the-needle peripheral intravenous catheter (PIVC). In this case, the catheter may be mounted over an introducer needle having a sharp distal tip. The catheter and the introducer needle may be assembled so that the distal tip of the introducer needle extends beyond the distal tip of the catheter with the bevel of the needle facing up away from the skin of the patient. The catheter and introducer needle are generally inserted at a shallow angle through the skin into the vasculature of the patient. After proper placement of the needle, the clinician may temporarily occlude flow in the vasculature and remove the needle, leaving the catheter in place (i.e., “indwelled”) for future blood withdrawal and/or fluid infusion.

In order to complete blood draws from PIVCs having indwelled catheters, blood draw devices have been developed that are configured to overcome previous challenges related to blood draw through PIVCs such as, e.g., the possibility of catheter collapse, reduced blood flow due to debris built up on or within the catheter, etc. Examples of such a device are shown and described in U.S. Patent Application Publication No. 2021/0290905 A1, which is incorporated herein by reference in its entirety. More recently, wheel-based blood draw devices have been provided with a physical stop configuration in order to demarcate a fully-advanced and fully-retracted position of the probe, while still enabling rotation of the wheel beyond a full 360° turn. Examples of such a device are provided in U.S. patent application Ser. No. 17/570,566, which is incorporated herein by reference in its entirety.

### SUMMARY OF THE INVENTION

According to one aspect or embodiment, an instrument advancement device for advancing an instrument through a vascular access device includes a housing having a distal end and a proximal end, with the distal end configured to couple to a vascular access device, an instrument at least partially received within the housing, with the instrument having an advanced position where a distal end of the instrument extends beyond the distal end of the housing and a retracted position where the distal end of the instrument is received within the housing, an actuator configured to move the instrument between the advanced position and the retracted position, and an indicator configured to be visible only when the instrument is in the advanced position.

The housing may include a viewing window, where the indicator is aligned with the viewing window when the instrument is in the advanced position. The indicator may include a first color or pattern and the housing may include a second color or pattern, where the first color or pattern is different than the second color or pattern.

The actuator may include an advancement member, with a portion of the advancement member moveable along an outer surface of the housing. The indicator may include indicia, where the indicia is covered by the advancement member when the instrument is in the retracted position and uncovered by the advancement member when the instrument is in the advanced position. The indicia may be positioned at the proximal end of the housing. The indicia may be positioned closer to the proximal end of the housing than the distal end of the housing.

The housing may include a viewing window, where the indicator includes a visualization portion of the instrument, and where the visualization portion of the instrument is aligned with the viewing window when the instrument is in the advancement position. The viewing window may include a magnification element. The instrument may be a guidewire, with the visualization portion of the instrument being a coiled portion of the guidewire. The coiled portion of the guidewire may have a compressed shape when the instrument is in the advanced position and a non-compressed shape when the instrument is in the retracted position. The visualization portion of the instrument may be a straight or cylindrical portion of the instrument. The visualization portion of the instrument may include a color, marking, and/or indicia distinct from a remaining portion of the instrument.

The indicator may be a light. The instrument may be a guidewire, where a portion of the guidewire is configured to activate the light when the instrument is moved to the advanced position.

The instrument advancement device may include an access connector positioned at the distal end of the housing, where the indicator is configured to move outside of the housing when the instrument is moved to the advanced position. The indicator may be configured to overlap the access connector when the instrument is moved to the advanced position.

The indicator may include an indicator member, where the indicator member is rotatable relative to the housing, and where the indicator member is configured to rotate to be visible when the instrument is in the advanced position.

Further details and advantages of the invention will become clear upon reading the following detailed description in conjunction with the accompanying drawing figures, wherein like parts are designated with like reference numerals throughout.

### BRIEF DESCRIPTION OF THE DRAWINGS

FIG. 1 is a front view of an instrument advancement device according to one aspect or embodiment of the present application, showing a retracted position of the device.

FIG. 2 is a front view of the instrument advancement device of FIG. 1, showing an advanced position of the device.

FIG. 3 is a partial front view of an instrument advancement device according to a further aspect or embodiment of the present application, showing a retracted position of the device.

FIG. 4 is a front view of the instrument advancement device of FIG. 3, showing an advanced position of the device.

FIG. 5 is a partial front view of an instrument advancement device according to a further aspect or embodiment of the present application, showing a retracted position of the device.

FIG. 6 is a front view of the instrument advancement device of FIG. 5, showing an advanced position of the device.

FIG. 7 is a front view of an instrument advancement device according to a further aspect or embodiment of the present application.

FIG. 8 is a partial front view of an instrument advancement device according to a further aspect or embodiment of the present application, showing an advanced position of the device.

FIG. 9 is a front view of the instrument advancement device of FIG. 5, showing a retracted position of the device.

FIG. 10 is partial cross-sectional view of an instrument advancement device according to a further aspect or embodiment of the present application.

FIG. 11 is a partial schematic view of an instrument advancement device according to a further aspect or embodiment of the present application.

FIG. 12 is a front view of an instrument advancement device according to a further aspect or embodiment of the present application.

FIG. 13 is a front view of an instrument advancement device according to a further aspect or embodiment of the present application.

### DESCRIPTION OF PREFERRED EMBODIMENTS

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user when the component or device is in the use position, i.e., when the user is holding a fluid transfer device in preparation for or during use. Similarly, in this application, the terms “in the distal direction” and “distally” mean in the direction toward the connector portion of the fluid transfer device, and the terms “in the proximal direction” and “proximally” mean in the direction opposite the direction of the connector.

While not shown or described herein, it is to be understood that the instrument advancement devices described below may be utilized in connection with any suitable vascular access device such as, e.g., the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable vascular access device.

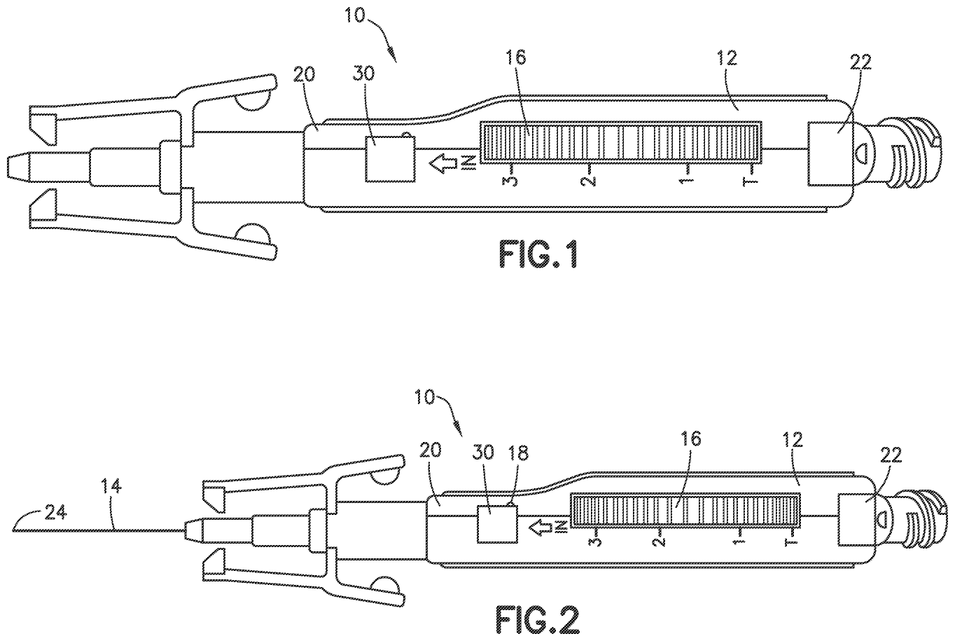

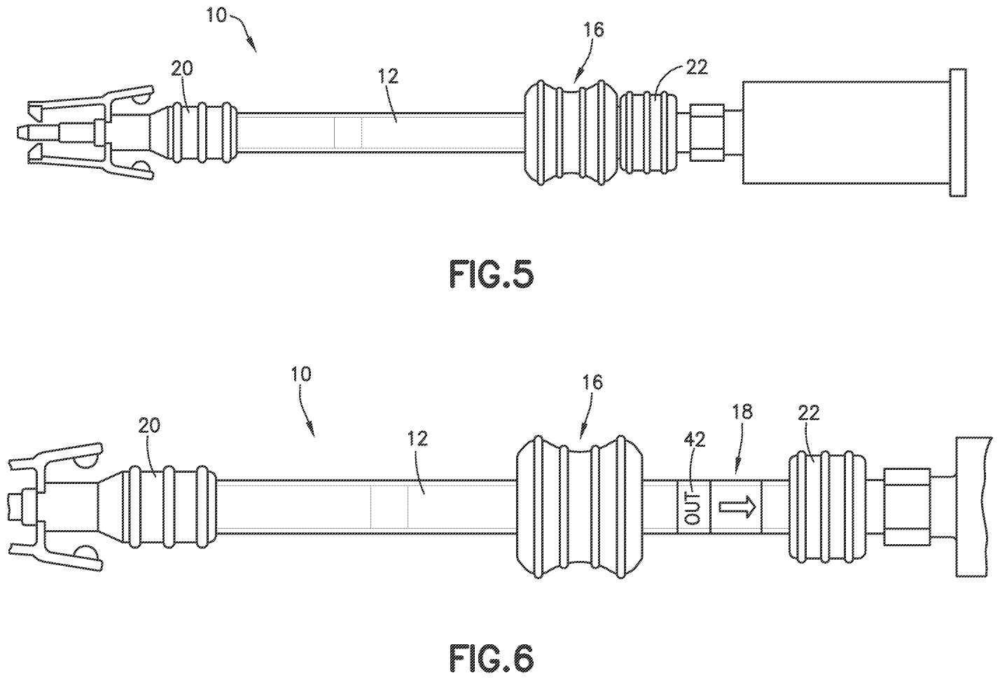

Referring to FIGS. 1-13, in one aspect or embodiment, an instrument advancement device 10 for advancing an instrument through a vascular access device includes a housing 12, an instrument 14 at least partially received within the housing 12, an actuator 16, and an indicator 18. The housing 12 has a distal end 20 and a proximal end 22, with the distal end 20 configured to couple to a vascular access device. The instrument 14 has an advanced position where a distal end 24 of the instrument 14 extends beyond the distal end 20 of the housing 12, and a retracted position where the distal end 20 of the instrument 14 is received within the housing 12. The actuator 16 is configured to move the instrument 14 between the advanced position and the retracted position. The indicator 18 is configured to be visible only when the instrument 14 is in the advanced position. The indicator 18 provides an indication to a healthcare worker that the instrument 14 is in the advanced position so that the device 10 is not removed from a vascular access device while the instrument 14 is in the advanced position. The advanced position of the instrument 14 is any position in which any part of the instrument 14 extends beyond the distal end 20 of the housing 12. The indicator 18 being visible only when the instrument 14 is in the advanced position includes the indicator 18 only being party or entirely visible. As detailed below, the indicator 18 may be utilized in connection with various styles of instrument advancement devices 10, including, but not limited to, linear advancement arrangements, flexible instrument arrangements, and wheel-based arrangements.

The instrument 14 may be a flow tube, a guidewire, a probe, a sensor, or other suitable arrangement. In some aspects or embodiments, the instrument 14 is configured to facilitate receiving a blood sample via a vascular access device placed within a vasculature of a patient by advancing the instrument to the advanced position.

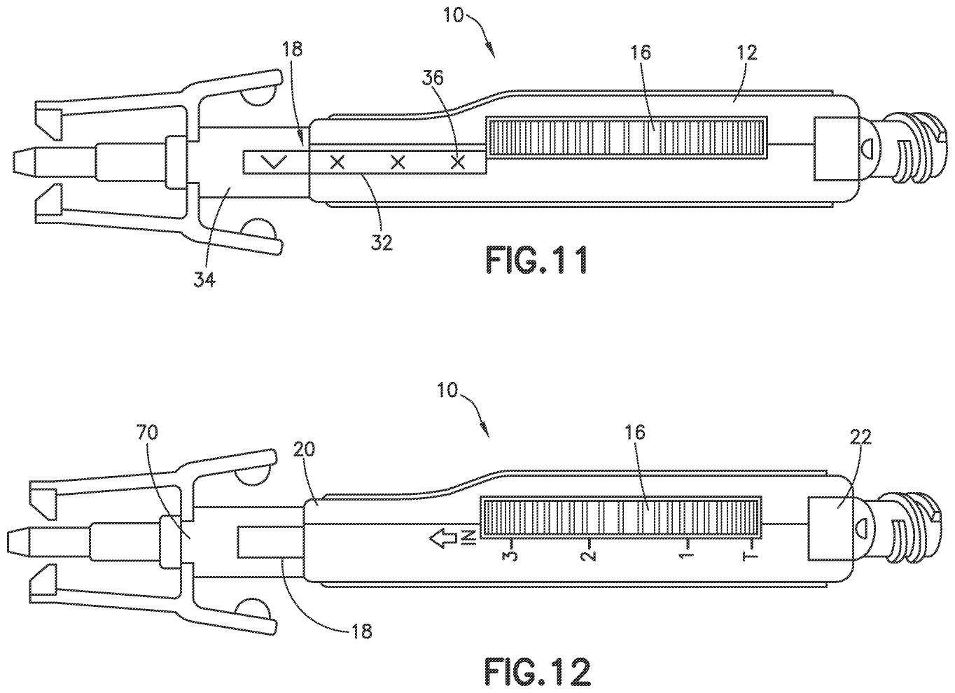

Referring to FIGS. 1, 2, and 11, in one aspect or embodiment, the housing 12 defines a viewing window 30, with the indicator 18 aligned with the viewing window 30 when the instrument 14 is in the advanced position. The indicator 18 may be positioned within the housing 12 and not visible through the viewing window 30 when the instrument 14 is in the retracted position. The indicator 18 is a first color or pattern and the housing 12 is a second color or pattern, with the first color or pattern different than the second color or pattern. The first color may be a dominant color compared to the rest of the instrument advancement device 10. In one aspect or embodiment, the first color is red or orange. The indicator 18 may include other indicia, such as symbols, words, or other markings. As shown in FIGS. 1, 2, and 11, the actuator 16 is a wheel-based arrangement and may be the same or similar to the devices shown in U.S. Patent Application Publication No. 2021/0290905 A1 and U.S. patent application Ser. No. 17/570,566. As shown in FIG. 11, the indicator 18 may be colored tape 32 having a first portion 34 visible via the viewing window 30 when the instrument 14 is in the retracted position and a second portion 36 visible via the viewing window 30 when the instrument 14 is in the advanced position.

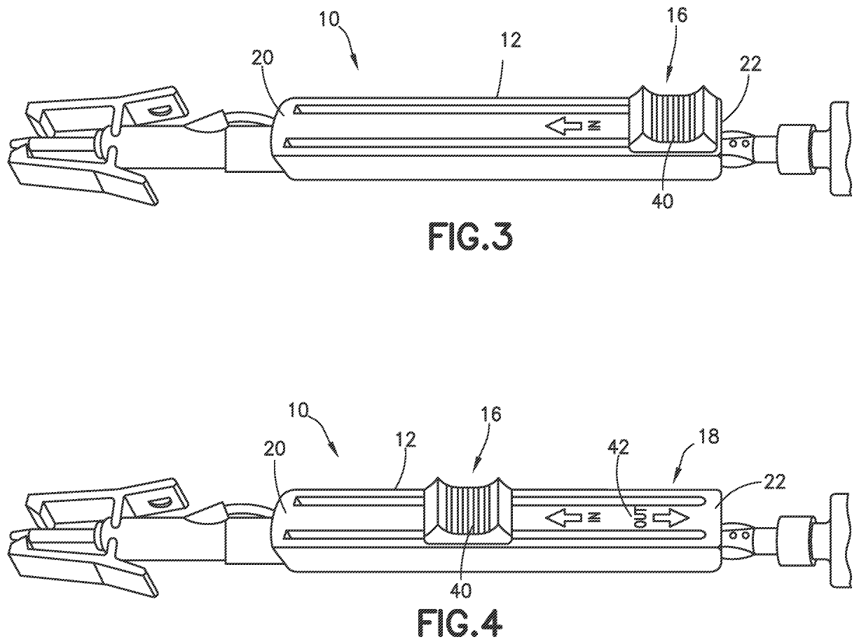

Referring to FIGS. 3-6, in one aspect or embodiment, the actuator 16 includes an advancement member 40, with a portion of the advancement member moveable along an outer surface of the housing 12. As shown in FIGS. 3 and 4, the actuator 16 may be the same or similar to the actuation arrangement shown and described in U.S. Pat. No. 11,090,461, which is incorporated by reference herein in its entirety. The indicator 18 includes indicia 42 that is covered by the advancement member 40 when the instrument 14 is in the retracted position and uncovered by the advancement member 40 when the instrument 14 is in the advanced position. In some aspects or embodiments, the indicia 42 is a symbol, words, or other markings. As shown in FIGS. 2-6, the indicia 42 is the word “OUT” with an arrow. The indicia 42 is positioned at the proximal end 22 of the housing 12. In some aspects or embodiments, the indicia 42 is positioned closer to the proximal end 22 of the housing 12 than the distal end 20 of the housing 12.

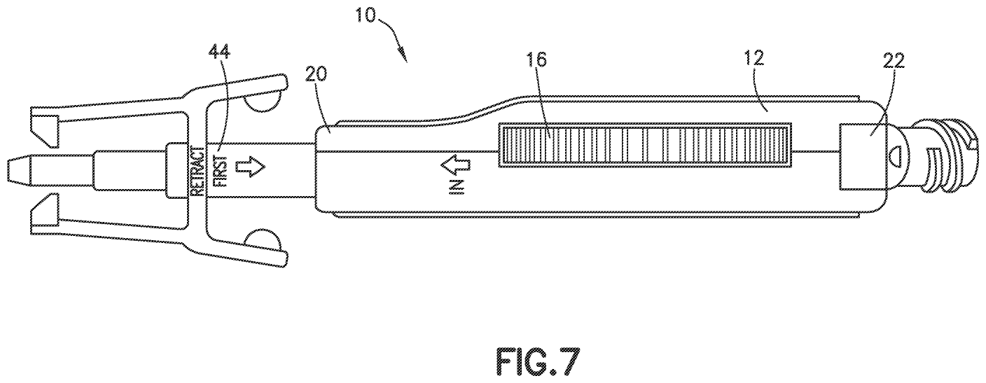

Referring to FIG. 7, in some aspects or embodiment, the instrument advancement device 10 includes secondary indicators 44 that are always visible to remind a healthcare worker to retract the instrument 14 prior to disconnecting the instrument advancement device 10 from a vascular access device.

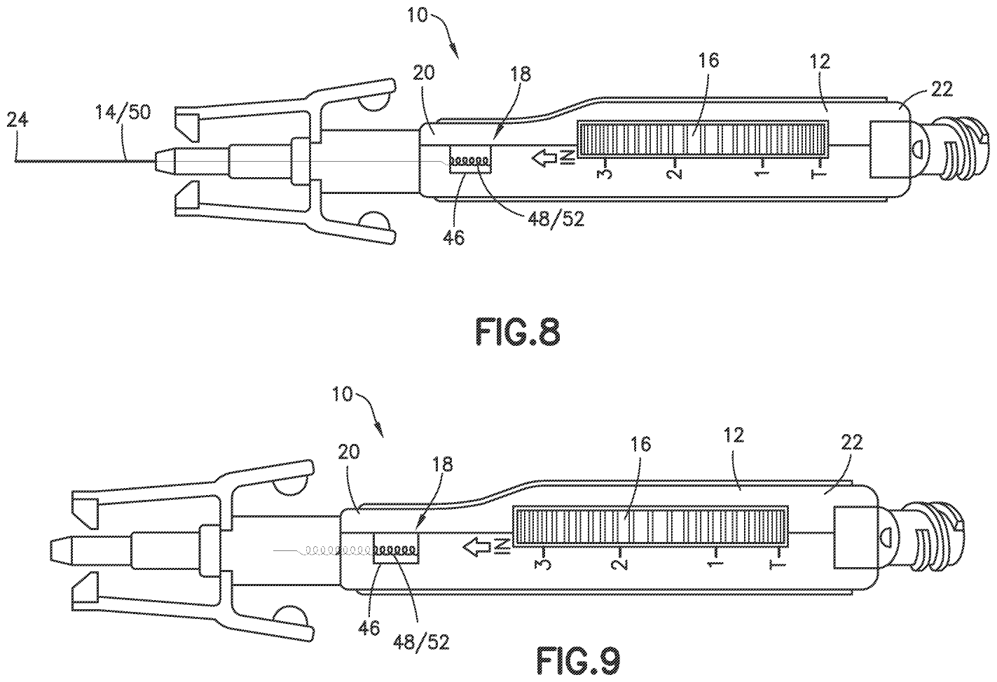

Referring to FIGS. 8 and 9, in one aspect or embodiments, the housing 12 defines a viewing window 46 and the indicator 18 is a visualization portion 48 of the instrument 14. The visualization portion 48 of the instrument 14 is aligned with the viewing window 46 when the instrument 14 is in the advanced position. In some aspects or embodiments, the viewing window 46 includes a magnification element (not shown), such as a magnification lens. As shown in FIGS. 8 and 9, the instrument 14 may be a guidewire 50 and the visualization portion 48 of the instrument may be a coiled portion 52 of the guidewire 50. The coiled portion 52 of the guidewire 50 has a compressed shape (shown in FIG. 8) when the instrument 14 is in the advanced position and a non-compressed shape (shown in FIG. 9) when the instrument 14 is in the retracted position. In some aspects or embodiments, the visualization portion 48 of the instrument 14 is a straight or cylindrical portion of the guidewire 50 when the instrument 14 is in the advanced position and a helical portion of the guidewire 50 when the instrument 14 is in the retracted position. The visualization portion 48 of the instrument 14 may include a color, marking, and/or indicia distinct from a remaining portion of the instrument 14. In some aspects or embodiments, the visualization portion 48 includes laser-etched markings on the instrument 14 or a specific grinding pattern on the instrument 14. The actuator 16 shown in FIGS. 8 and 9 may be a wheel-based arrangement and may be the same or similar to the devices shown in U.S. Patent Application Publication No. 2021/0290905 A1 and U.S. patent application Ser. No. 17/570,566.

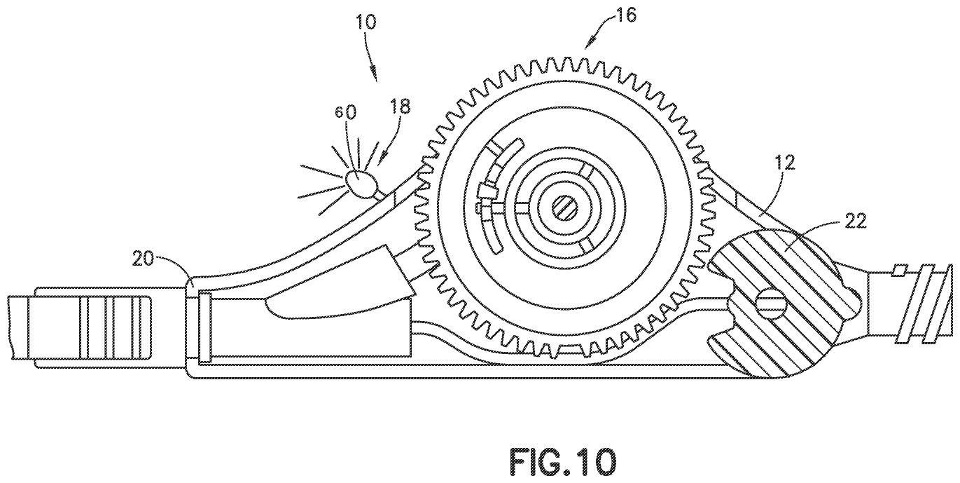

Referring to FIG. 10, in one aspect or embodiment, the indicator 18 is a light 60. The instrument 14 may be a guidewire, with a portion of the guidewire configured to activate the light 60 when the instrument 14 is moved to the advanced position. In some aspects or embodiments, the actuator 16, wheels or other portions of the actuator 16, or other mechanisms or portions of the instrument advancement device 10 not shown. In one aspect or embodiment, the guidewire may have a larger diameter portion that completes a circuit to illuminate the light 60 when the instrument 14 is moved to the advanced position. In some aspects or embodiments, the secondary wheel near the hub of the actuator 16, such as a wheel of the actuator 16, may complete a circuit when the forward advancement is completed and an arm between the actuator 16 and the indicator 18 snaps into a detent near the light 60. The actuator 16 shown in FIG. 10 may be a wheel-based arrangement and may be the same or similar to the devices shown in U.S. Patent Application Publication No. 2021/0290905 A1 and U.S. patent application Ser. No. 17/570,566. The actuator 16 may include a part that moves when the wheel is advanced to complete a circuit to illuminate the light 60. The light 60 may be an LED light and may be powered by a battery.

Referring to FIG. 12, in one aspect or embodiment, the instrument advancement device 10 includes an access connector 70 positioned at the distal end 20 of the housing 12, with the indicator 18 configured to move outside of the housing 12 when the instrument 14 is moved to the advanced position. The indicator 18 is configured to overlap the access connector 70 when the instrument 14 is moved to the advanced position. By overlapping the access connector 70, the indicator 18 will be readily apparent to a healthcare worker when grasping the access connector 70 to remove the device 10 from a vascular access device thereby ensuring the instrument 14 is moved to the retracted position prior to disconnecting the device 10.

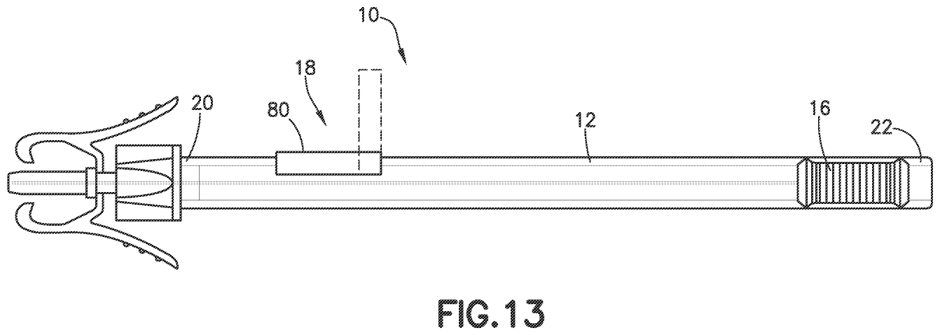

Referring to FIG. 13, in one aspect or embodiment, the indicator 18 includes an indicator member 80 rotatable relative to the housing 12. The indicator member 80 is configured to rotate to be visible when the instrument 14 is in the advanced position. The actuator 16 may be the same or similar to the actuation arrangement shown and described in U.S. Pat. No. 11,090,461. As the actuator 16 is advanced to the forward position, the actuator 16 engages the indicator member 80 and rotates the indicator member 80 such that a portion of the indicator member 80 is visible or positioned outside of the housing 12. The indicator member 80 is configured to rotate again such that the indicator member 80 is not visible or positioned outside of the housing 12 when the actuator 16 is moved backward with the instrument 18 in the retracted position.

While several embodiments of blood draw and delivery devices configured for blood draw during catheter indwell were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

What is claimed is:

1. An instrument advancement device for advancing an instrument through a vascular access device, the device comprising:
a housing comprising a distal end and a proximal end, the distal end is configured to couple to the vascular access device;
an instrument at least partially received within the housing, the instrument having an advanced position where a distal end of the instrument extends beyond the distal end of the housing and a retracted position where the distal end of the instrument is received within the housing;
an actuator configured to move the instrument between the advanced position and the retracted position; and
an indicator configured to be visible only when the instrument is in the advanced position.

2. The device of claim 1, wherein the housing comprises a viewing window, wherein the indicator is aligned with the viewing window when the instrument is in the advanced position.

3. The device of claim 2, wherein the indicator comprises a first color or pattern and the housing comprises a second color or pattern, and wherein the first color or pattern is different than the second color or pattern.

4. The device of claim 1, wherein the actuator comprises an advancement member, a portion of the advancement member moveable along an outer surface of the housing, wherein the indicator comprises indicia, and wherein the indicia is covered by the advancement member when the instrument is in the retracted position and uncovered by the advancement member when the instrument is in the advanced position.

5. The device of claim 4, wherein the indicia is positioned at the proximal end of the housing.

6. The device of claim 4, wherein the indicia is positioned closer to the proximal end of the housing than the distal end of the housing.

7. The device of claim 1, wherein the housing comprises a viewing window, wherein the indicator comprises a visualization portion of the instrument, and wherein the visualization portion of the instrument is aligned with the viewing window when the instrument is in the advanced position.

8. The device of claim 7, wherein the viewing window comprise a magnification element.

9. The device of claim 7, wherein the instrument comprises a guidewire, and wherein the visualization portion of the instrument comprises a coiled portion of the guidewire, the coiled portion of the guidewire having a compressed shape when the instrument is in the advanced position and a non-compressed shape when the instrument is in the retracted position.

10. The device of claim 7, wherein the visualization portion of the instrument comprises a straight or cylindrical portion of the instrument.

11. The device of claim 7, wherein the visualization portion of the instrument comprises a color, marking, and/or indicia distinct from a remaining portion of the instrument.

12. The device of claim 1, wherein the indicator comprises a light.

13. The device of claim 12, wherein the instrument comprises a guidewire, and wherein a portion of the guidewire is configured to activate the light when the instrument is moved to the advanced position.

14. The device of claim 1, further comprising an access connector positioned at the distal end of the housing, wherein the indicator is configured to move outside of the housing when the instrument is moved to the advanced position.

15. The device of claim 14, wherein the indicator is configured to overlap the access connector when the instrument is moved to the advanced position.

16. The device of claim 1, wherein the indicator comprises an indicator member, wherein the indicator member is rotatable relative to the housing, and wherein the indicator member is configured to rotate to be visible when the instrument is in the advanced position.
